(19) United States
(12) Patent Application Publication
(10) Pub. No.: US 2024/0032672 A1
(43) Pub. Date: Feb. 1, 2024

(54) APPLICATOR SYSTEMS

(71) Applicant: JALLATI Inventions, LLC, Vienna, VA (US)

(72) Inventor: Layne Summerfield KROH, Vienna, VA (US)

(73) Assignee: JALLATI Inventions, LLC, Vienna, VA (US)

(21) Appl. No.: 18/358,595

(22) Filed: Jul. 25, 2023

Related U.S. Application Data
(60) Provisional application No. 63/369,792, filed on Jul. 29, 2022.

(51) Int. Cl. A45D 34/04

(52) U.S. Cl. CPC A45D 34/046; A45D 2200/25; A45D 2200/05

(57) ABSTRACT

Applicator systems and kits for applying a product to hair or skin as well as methods of use thereof are described. The applicator system may include a brush including a handle and a brush head; a cap configured to couple to a portion of the brush; a refillable container including an opening; and a divider including an opening for receiving the brush and removing excess product from the brush. The divider may be attached to the opening of the refillable container.

### CROSS-REFERENCE TO RELATED APPLICATION(S)

This application claims priority to U.S. Provisional Application No. 63/369,792 filed on Jul. 29, 2022, which is incorporated by reference herein in its entirety.

### TECHNICAL FIELD

Various embodiments of the present disclosure relate generally to applicator systems and related methods of use thereof and, more particularly, to brush applicator systems for applying a variety of products and removing excess product from the brush.

### BACKGROUND

Brushes of various sizes and shapes are used to apply a variety of products to the skin and hair. In various instances of applying a product with a brush, the brush used for product application is often sold and stored separately from the product that is applied. When applying products such as makeup (e.g., foundation, bronzer, blush, concealer, etc.), a different brush may be required for each product, which often results in a cumbersome amount of items for a user to carry and keep track of. Storing brushes separately from a product for application may also increase the risks of losing and even damaging the brushes. In addition, applying too much of a product to a brush may cause a mess for the user and result in waste of the product. Therefore, a need exists for a compact system for storing brushes and products, which can also remove excess product from the brush.

### SUMMARY OF THE DISCLOSURE

The present disclosure includes applicator systems and kits for applying a product to hair or skin. For example, the present disclosure includes an applicator system. The applicator system may include a brush including a handle and a brush head; a cap configured to couple to a portion of the brush; a refillable container including an opening; and a divider, the divider including an opening for receiving the brush and removing excess product from the brush. The divider may be attached to the opening of the refillable container. In a first state, the brush may be housed in the refillable container and the cap may be removably attached to the refillable container. In a second state, a brush head of the brush may include a product stored in the refillable container and may be in contact with a portion of the divider. In some examples, the divider comprises a flexible material. In at least one example, the refillable container has a volume greater than 15 mL. In some examples, the brush includes a brush head and the opening of the divider may have a diameter that is greater than a width of the brush head.

In at least one example, a portion of the divider may extend radially into a depth of the refillable container. The opening of the divider may have a diameter ranging from about 10 mm to about 100 mm. In some aspects of the present disclosure, the refillable container may be filled with a product and the product may be selected from a sunscreen, self-tanner, moisturizer, lotion, body wash, facial wash, cleansing cream, serum, exfoliator, toner, peel, mask, body oil, hair gel, hair dye, rubbing alcohol, hydrogen peroxide, medical ointment, eye shadow, bronzer, concealer, blush, lip treatment, or foundation. In some examples herein, the cap may be a screw top, snap top, or pop-top. In certain examples, the divider may be fixed to the refillable container. In other examples, the divider may be detachable from the refillable container. In at least one example, both the refillable container and the divider may comprise a flexible material. In certain examples herein, the opening of the divider may be adjustable.

Another aspect of the present disclosure may include an applicator system. The applicator system may include a brush; a cap configured to couple to a portion of the brush; and a container for storing the brush and filling with a product for application to the brush. The container may hold a volume of at least 20 mL of the product and the cap may attach to an opening of the container to store the brush in the container and provide an airtight seal. In some examples, the brush may be interchangeable with a different brush for storing in the container. In at least one example, the brush may be detachable from the cap.

In some examples herein, the applicator system may further comprise a divider attached to the opening of the container and positioned between the cap and the container. The divider may include an opening sized for insertion of the brush into the container and removal of the brush from the container. In some aspects, the divider may be configured to remove an amount of the product form the brush when the brush is removed from the container. In at least one example, both the container and the divider may comprise a flexible material. In at least one example, the opening of the divider may be adjustable. In some examples, the container may be filled with sunscreen or a medicament.

Another aspect of the present disclosure may include a kit for applying a product to hair or skin. The kit may include: a container for filling with a product and storing a brush for applying the product inside of the container; a brush for applying the product; and a divider for attaching to the container. The divider may include an opening for receiving a brush and removing excess product from a brush. In some examples herein, the kit may include a plurality of brushes and dividers.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

There are many inventions described and illustrated herein. The described inventions are neither limited to any single aspect nor embodiment thereof, nor to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 illustrates a schematic exemplary applicator system with a brush cap according to one or more embodiments of the disclosure.

FIG. 2 illustrates an exploded view of an exemplary applicator system according to one or more embodiments of the disclosure.

FIG. 3 illustrates an exemplary kit according to one or more embodiments of the disclosure.

FIGS. 4A-4G illustrate exemplary dividers according to one or more embodiments of the disclosure.

FIGS. 5A-5B illustrate an exemplary applicator system according to one or more embodiments of the disclosure.

FIGS. 6A-6B illustrate an exemplary applicator system according to one or more embodiments of the disclosure.

FIGS. 7A-7B illustrate an exemplary applicator system according to one or more embodiments of the disclosure.

FIGS. 8A-8D illustrate exemplary brushes according to one or more embodiments of the disclosure.

FIGS. 9A-9B illustrate another exemplary applicator system according to one or more embodiments of the disclosure.

### DETAILED DESCRIPTION OF EMBODIMENTS

The terminology used herein may be interpreted in its broadest reasonable manner, even though it is being used in conjunction with a detailed description of certain specific examples of the present disclosure. Indeed, certain terms may even be emphasized herein; however, any terminology intended to be interpreted in any restricted manner will be overtly and specifically defined as such in this Detailed Description section. Both the general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

Spatially relative terms, such as “top,” “bottom,” “middle,” “inner,” “outer,” “beneath,” “below,” “lower,” “above,” “upper,” and the like, may be used herein for ease of description to describe one element or feature's relationship to another element(s) or feature(s) as illustrated in the drawings. Spatially relative terms may be intended to encompass different orientations of a device in use or operation in addition to the orientation depicted in the drawings. For example, if the device in the drawings is turned over, elements described as “below” or “beneath” other elements or features would then be oriented “above” the other elements or features.

As discussed above, a variety of cosmetic products may be applied to various parts of the body, e.g., the hair, face, neck, arms, legs, stomach, and feet. Such cosmetic products may include sunscreen, self-tanner, moisturizers, lotions, body wash, facial wash, cleansing cream, serum, exfoliator, face oil, face masks, toners, peels, body masks, body oil, body powder, hair gel, hair dye, perms, relaxers, chemical hair straighteners, eye shadow, bronzer, highlighter, concealer, blush, lip treatment, face powder, face paint, and foundation. The products may be stored in a container (e.g., a bottle) that is suitable for the respective product. Expelling the product from the container and directly onto the body may cause an uncontrolled amount of the product to be released onto the hands, which may create a mess for the user and result in the waste of excess product. In some instances, the product may also be difficult to apply to the body when dispensed directly from the container.

A common method for applying products (e.g., cosmetic products) to the body includes using a single applicator (e.g., a brush) or a plurality of applicators to apply the product. However, oftentimes the applicator used for product application is sold and stored separately from the container that includes the product. Having to store one or more applicators separately from the container(s) comprising the product for application results in a cumbersome amount of items for a user to carry and keep track of, especially when traveling. Further, when brushes are used, the bristles of the brush may retain excess product, resulting in further product waste.

Thus, a need exists for systems configured to store applicators and products for applying with the applicator, which may also remove excess product from the applicator. The embodiments of the present disclosure are directed to solving, mitigating, or rectifying the above-mentioned issues by providing a compact device for storing both a variety of products and a brush for applying the product, removing excess product from a brush, reducing product waste, and providing a cleaner method of applying traditionally messy products to the body.

Although the present disclosure references personal or cosmetic products, those of ordinary skill in the art will readily recognize that the concepts of the present disclosure may be utilized with any suitable or comparable products.

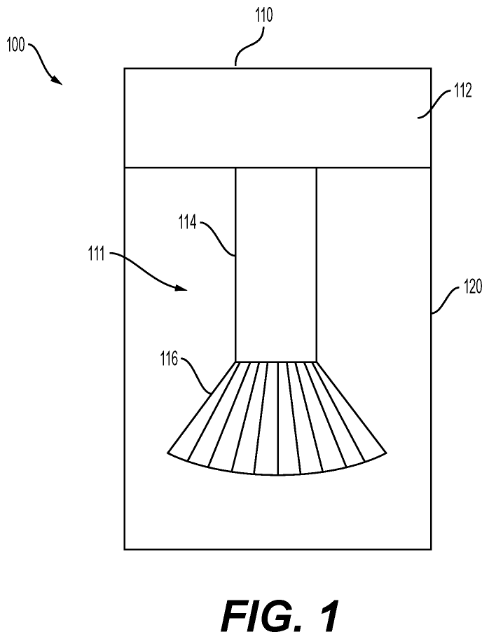

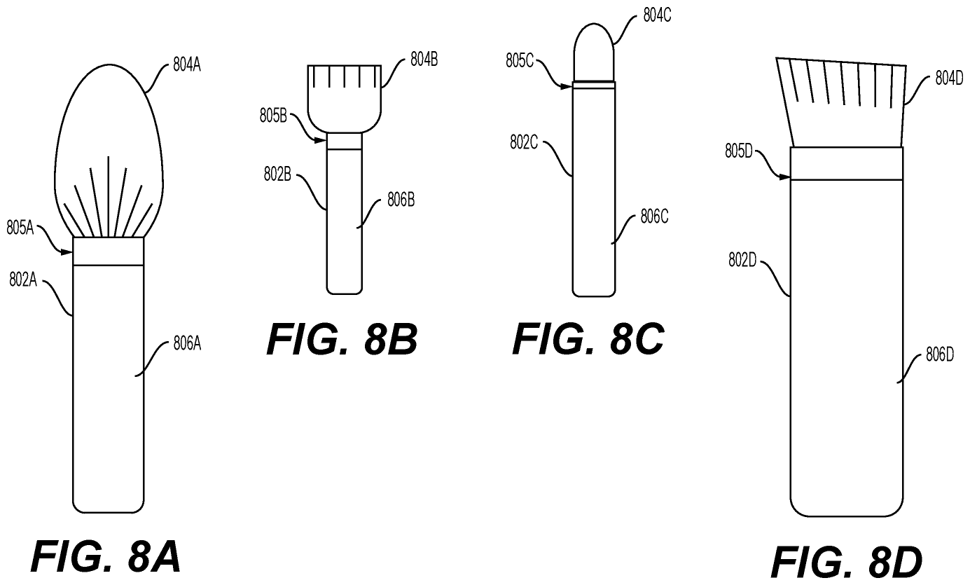

Embodiments of the present disclosure relate generally to applicator systems, and in particular, to applicator systems for applying a variety of products to the body, e.g., hair or skin. FIG. 1 depicts an exemplary applicator system 100. As shown in FIG. 1, applicator system 100 may include a brush cap 110 and a container 120. The brush cap 110 may include a brush 111 and a cap 112. The brush 111 may include a handle 114 and a brush head 116. The brush head 116 may include a plurality of bristles, and the brush head 116 may be attached to the handle 114 at one end through a ferrule (not shown). The brush 111 of applicator system 100 may be of any size and shape as discussed in further detail below in reference to FIGS. 8A-8D. The handle 114 of the brush 111 may be attached to the cap 112 at the end of the handle 114 that is opposite the end connected to the brush head 116.

The cap 112 may comprise a variety of materials. For example, the cap 112 may comprise plastic, wood, recyclable materials, steel, aluminum, or other metals. The cap 112 may be configured to attach to the container 120. The cap 112 may have various configurations for engaging with and attaching to the container 120. The cap 112 may be in the form of a screw top cap, a snap on cap, or a push-pull cap. A screw top cap may include threads for screwing the cap 112 onto the container 120. A snap cap may be configured to snap onto the container 120. For example, a snap cap may be configured to surround the neck of the container 120. A push-pull cap may be configured to push or insert a lower portion of the cap 112 into the container 120 and to pull or remove the lower portion of the cap 112 out of the container 120. In some examples, the cap 112 may be child resistant. In configurations where the cap 112 is engaged with and attached to the container 120, the cap 112 may prevent the contents of the container 120 from spilling.

The container 120 may be a refillable container. For example, the container 120 may be filled with one or more products as desired by the user. The container 120 may be made from a variety of materials. For example, the container 120 may be made from plastic, wood, recyclable materials, or any other appropriate material for storing personal products, e.g., cosmetic products. In some examples, the container 120 may be made from a flexible material. For example, the container 120 may be made from flexible plastic, rubber, or silicone material. Exemplary flexible materials may include neoprene, butyl rubber, nitrile rubber, silicone rubber, polyvinyl chloride (PVC), polyethylene and polypropylene. In certain examples herein, the container 120 may be manufactured from a flexible material to aid in controlling the amount of product, e.g., cosmetic product that is retained by the brush head 116 as or prior to the brush 111 used to obtain a product being stored in the container 120 is removed from the container 120. When the container 120 comprises a flexible material, pressure or force may be applied to the container 120, such as by squeezing. The application of pressure (e.g., squeezing) to the container 120 made of a flexible material may cause inner walls of the container 120 to compress and for internal volume to decrease, such that the shape of the container 120 is altered (e.g., deformed) and a passageway within the container 120 is narrowed. For example, squeezing the container 120 may allow inner surfaces of the container 120 to press against the brush head 116 in order to remove excess product. After squeezing the container 120, the flexible material from which the container 120 is made may allow the container 120 to return to its original shape. In some examples, excess product removed from the brush head 116 by squeezing the container 120 may be retained on an inner surface of the container 120.

The container 120 may be transparent and may allow the contents to be viewable through the container 120 as shown in FIG. 1. Alternatively, the container 120 may be semi-transparent or opaque.

The container 120 may have any size and/or shape appropriate for properly storing products, such that applicator system 100 may be portable and easily movable. For example, containers utilized in the embodiments discussed herein may have a circular shape, a cylindrical shape, a triangular shape, or a rectangular shape. In some examples, the container 120 may have a cylindrical shape, similar to a bottle. The size, shape, and/or material of the container 120 may vary based on the product in the container 120. In some examples, the container 120 may have a volume of at least 15 milliliters (mL). For example, the container 120 may be configured to hold a volume of at least 15 mL of a product, at least 20 mL of a product, or at least 30 mL of a product. Exemplary volumes for container 120 may range from 10 mL to 500 mL, from 15 mL to 100 mL, from 20 mL to 75 mL, 25 mL to 50 mL, or from 30 mL to 40 mL. A height of the container 120 may range from about 75 mm to about 250 mm. For example, a height of the container 120 may range from about 75 mm to about 220 mm, about 85 mm to about 200 mm, about 90 mm to about 175 mm or about 95 mm to about 150 mm. A width, e.g., a diameter or a cross-sectional dimension, of the container 120 range from about 40 mm to about 125 mm. For example, the container 120 may have a width ranging from about 50 mm to about 100 mm, about 50 mm to about 90 mm, about 50 mm to about 75 mm, or about 55 mm to about 65 mm.

As shown in FIG. 1, the brush 111 of the brush cap 110 may be stored in the container 120 of applicator system 100. In other words, when the brush cap 110 is attached to the container 120, a portion of the brush 111 may be fully enclosed inside the container 120. For example, a portion of the handle 114 and the brush head 116 may be fully enclosed inside the container 120. The container 120 may have a height or depth that allows the entire brush 111 of the brush cap 110 to fit inside of the container 120 when the cap 112 is attached to the container 120. The container 120 may have various configurations for engaging with the cap 112. In some examples, the top or neck portion of the container 120 may include threads for allowing the cap 112 to be screwed or twisted on and off the container 120. The top or neck portion of the container 120 may be sized to allow the cap 112 to cover the opening of the container 120.

Applicator system 100 may be airtight. The cap 112 may provide an airtight seal when it is attached to the container 120. In addition to enclosing or sealing the brush 111 of the brush cap 110 inside of the container 120, the cap 112 may also seal the contents (e.g., product) inside of the container 120 until the cap 112 is detached from the container 120. The airtight seal provided by the cap 112 of the brush cap 110 may prevent the contents in the container 120 from drying out.

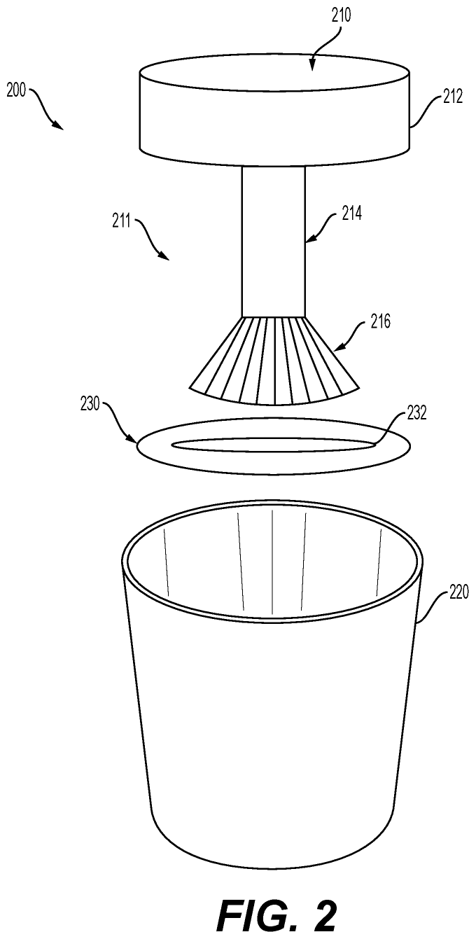

FIG. 2 depicts an exploded view of an exemplary applicator system 200. The exploded view shown in FIG. 2, illustrates the components that make up an applicator system 200 and the relative positions of each component when the applicator system 200 is in an assembled state. The applicator system 200 may include a brush cap 210 and a container 220. The brush cap 210 and the container 220 may have the same features as those described for the brush cap 110 and the container 120 of applicator system 100. For example, the brush cap 210 may include a brush 211 and a cap 212. The brush 211 may include a handle 214 and a brush head 216. The handle 214 of the brush 211 may be attached to the cap 212 at the end of the handle 214 that is opposite the end connected to the brush head 216. The cap 212 of the brush cap 210 may attach to the container 220 and the brush 211 of the brush cap 210, such that a portion of the brush cap 210, e.g., a portion of the handle 214 and the brush head 216, may extend into the container 220. As discussed above, the container 220 may house the brush 211 of the brush cap 210.

Applicator system 200 may include a divider 230. The divider 230 may be made from plastic, metal, rubber, sponge, or a synthetic packaging material. In some examples, the divider 230 may be made from the same material as the container 220 and/or the cap 212 of the brush cap 210. The divider 230 may be located proximal to the opening of the container 220. For example, the divider 230 may be attached at the opening of the container 220. In some examples, the divider 230 may be placed on top of the container 220 over the opening of the container 220 leading into the container 220. The divider 230 may include an opening 232 for receiving and/or dispensing a portion of brush cap 210, e.g., handle 214 and/or brush head 216.

The divider 230 and the opening 232 of divider 230 may be configured to facilitate free movement of the brush 211 into the container 220 and out of the container 220 without damaging the brush head 216 and bristles contained therein. The opening 232 of the divider 230 may have a diameter that is greater than the width of the brush head 216. In some examples, a diameter of the opening 232 of the divider 230 may be greater than the width of the brush 211 of the brush cap 210, including both the handle 214 and the brush head 216, to allow the handle 214 and the brush head 216 to be pulled through the opening 232 of the divider 230 with ease. The opening 232 of the divider 230 may have a diameter that exceeds the width of the brush head 216 of the brush 211, to help remove excess product from the brush 211 prior to and/or during removal of the brush 211 from the container 220. In some examples herein, as the brush 211 of the brush cap 210 is pulled through the divider 230, in a direction away from the container 220, edges (or an inner rim) at the opening 232 of the divider 230 may be used to wipe or otherwise retain excess product contained on the brush head 216. The divider 230 may have a diameter or cross-sectional dimension ranging from about 40 mm to about 125 mm. For example, the divider 230 may have a diameter or cross-sectional dimension ranging from about 50 mm to about 100 mm, about 50 mm to about 90 mm, about 50 mm to about 75 mm, or about 55 mm to about 65 mm. The opening 232 of the divider 230 may have a diameter ranging from about 10 mm to about 100 mm. For example, the opening 232 of the divider 230 may have a diameter ranging from about 10 mm to about 75 mm, about 10 mm to about 50 mm, about 10 mm to about 25 mm, about 25 mm to about 100 mm, or about 50 mm to about 100 mm.

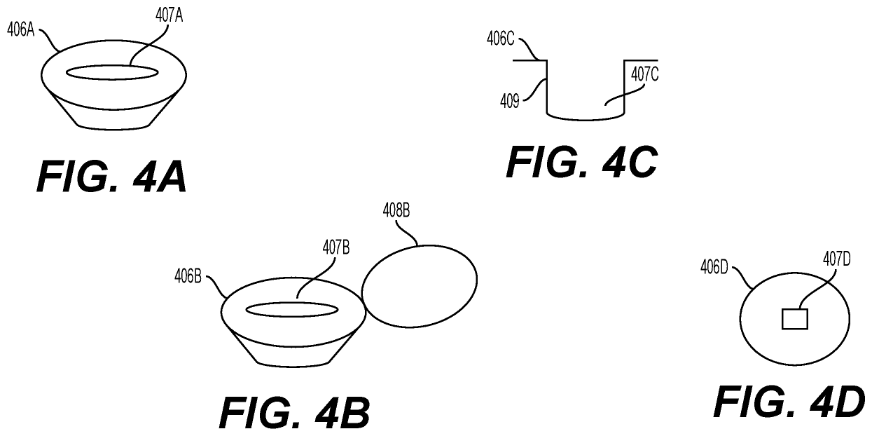

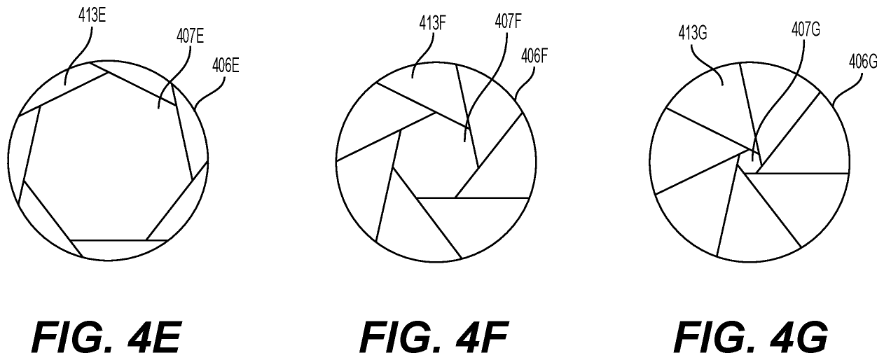

In at least one example, the divider 230 may be a stationary component that may be permanently affixed to the container 220. In other examples, the divider 230 may be a removable component. The divider 230 may be a disposable component that may be discarded after each use. For example, the divider 230 may be configured to snap onto the container 220, such that the divider 230 may be snapped off, e.g., popped off, in order to remove the divider 230 from the container 220. In other examples, the divider 230 may have threads for screwing or twisting the divider 230 on and off the container 220. A more detailed description of dividers according to the present disclosure is provided below in reference to FIGS. 4A-4D.

The exploded view of exemplary applicator system 200 also depicts how applicator systems of the present disclosure may be assembled. For example, the container 220 may be obtained and filled with a product. The divider 230 may be attached to the container 220, e.g., by snapping the divider 230 onto the container 220. Next, the brush 211 of brush cap 210 may be inserted through the opening 232 of the divider 230. The brush 211 of the brush cap 210 may continue to be inserted into the container 220 through the opening 232 of the divider 230, until the cap 212 of the brush cap 210 contacts the divider 230. Once the brush 211 of the brush cap 210 is fully housed within the container 220, the cap 212 of the brush cap 210 may be attached to the container 220 in a manner consistent with the type of cap 212 (e.g., snap top, screw top, etc.) to form an airtight seal. The applicator system 200 may be disassembled by removing the components in the order the order they were attached to the container 220. For example, the applicator system 200 may be disassembled by detaching the cap 212 of the brush cap 210 from the container 220 and lifting the brush cap 210 through the opening 232 of the divider 230. Next, the divider 230 may be detached from the container 220 and removed.

Another method of assembling exemplary applicator system 200 may include attaching the brush cap 210 to the container 220 prior to the attachment of the divider 230. In this method, the brush cap 210 may be detached from the container 220 and the brush 211 may be removed. The divider 230 may then be attached to the container 220. In some examples, the product may be added to the container 220 before the divider 230 is added. In other examples, the opening 232 of the divider 230 may be configured to receive the product as it is filled into the container 220 through the opening 232 of the divider 230. Next, the brush 211 of the brush cap 210 may be inserted through the opening 232 of the divider 230 and the cap 212 of the brush cap 210 may be attached to the container 220 in a manner consistent with the type of cap 212 used for the brush cap 210 to form an airtight seal. In certain examples, the divider 230 may be a built-in component of the container 220. In these examples, the container 220 may be filled with a product through the opening 232 of the divider 230. The brush 211 of the brush cap 210 may be inserted through the opening 232 of the divider 230 and the cap 212 may be attached to container 220 in a manner consistent with the cap type.

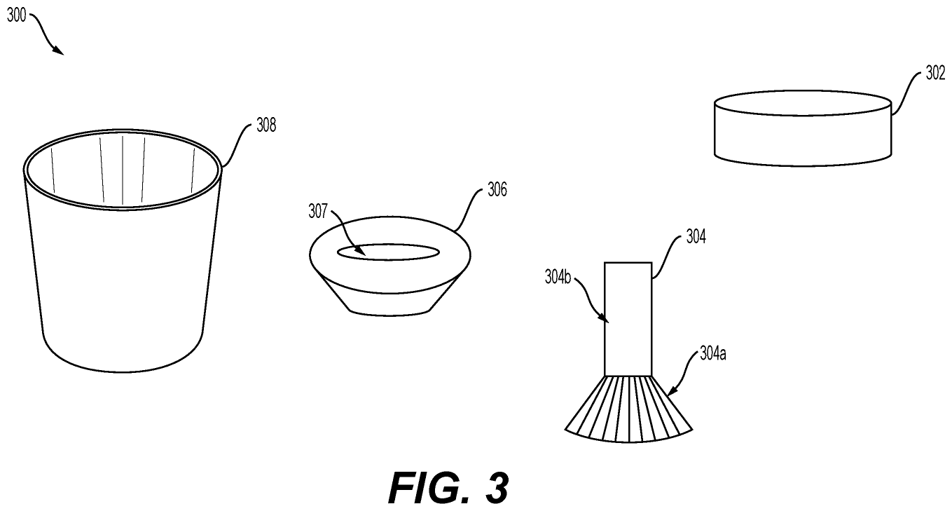

FIG. 3 depicts an exemplary kit 300. Kit 300 may contain separate components of an applicator system. For example, the kit 300 may include a cap 302, a brush 304, a divider 306, and a container 308. Each of the cap 302, the brush 304, the divider 306, and the container 308 may have the same features as those described for the respective components in applicator systems 100 and 200. In some examples, the kit 300 may contain a plurality of each component. In other examples, the kit 300 may include a subset of the components shown in FIG. 3.

The cap 302 may be configured to attach to the container 308. The cap 302 may be selected from a screw top cap, a snap on cap, a child-resistant cap, or a push-pull cap. In examples, where the kit 300 includes a plurality of cap 302, the kit 300 may include various combinations of the caps listed above. The cap 302 may further be configured to attach to the brush 304. For example, the cap 302 may include a receiving portion (not shown) for coupling with the brush 304.

While FIG. 3 depicts one brush 304, one or more brushes may be included in the kit 300. In some examples, the brush 304 may be configured for attachment to the cap 302. Each brush 304 in the kit 300 may be configured to attach to a single cap 302. When more than one brush 304 is included in the kit 300, each of the brushes may have varying sizes and/or shapes. The size of the brush 304 may include the total length of the brush 304 from a first end to a second end, including the length of a brush handle 304b and the length of the brush head 304a. For example, the brush 304 may have a total length ranging from 50 mm to 200 mm. The total length of the brush 304 may vary based on the height of the container 308. In some examples, the brush 304 may have a total length that allows the entire brush 304 to fit inside of the container 308 when the brush 304 is attached to the cap 302 and the cap 302 is attached to the container 308. In these examples, the total length of the brush 304 may be less (e.g., shorter) than the height of the container 308. In some examples, the total length of the brush 304 may be equal to or substantially equal to the height of the container 308. In other examples, the brush handle 304b may extend outside of the container 308 when the brush 304 is fully inserted into the container 308, such that the brush head 304a is near the bottom of the container 308. For example, the total length of the brush 304 may be greater than the height of the container 308. In examples herein, a ratio of the total length of the brush 304 to the height of the container 308 may range from 1:3 to 3:1. An exemplary ratio of the total length of the brush 304 to the height of the container 308 may be 1:2, 2:3, 4:5, 3:2, 2:1, or 8:3. The width of the brush head 304a may range from 5 mm to 75 mm. Each brush head 304a may also have a different shape (e.g., round, angled, tapered, etc.).

While FIG. 3 depicts one divider 306, one or more dividers may be included in the kit 300. The divider 306 may be configured to attach to the opening of the container 308. The divider 306 may also have an opening 307 configured to receive the brush 304. The opening 307 of the divider 306 may have a diameter that is greater than the width of the brush head 304a of the brush 304. Exemplary diameters of the opening 307 of the divider 306 may range from about 10 mm to about 100 mm. The size or diameter of the opening 307 of the divider 306 may vary based on the viscosity of the product being used with the divider 306. More than one divider 306 may be included in the kit 300 and each divider 306 may be configured for use with a different type of intended product.

The container 308 may be configured to form an attachment with the cap 302. The container 308 may also be configured to form an attachment with the divider 306. In at least one example, a plurality of container 308 may be included in the kit 300. When more than one container 308 is included in the kit 300, each of the containers may have varying sizes and/or shapes. The size of each container 308 may correspond to the quantity of the product for storing in the container 308 as well as to the size of each cap 302 and/or each divider 306 included in the kit 300. The height and width of the container 308 may vary based on the total length of the brush 304 and the width and/or shape of the brush head 304a of the brush 304. A length of container 308 may range from about 75 mm to about 220 mm. For example, a length of container 308 may range from about 75 mm to about 200 mm, about 85 mm to about 175 mm, or about 95 mm to about 150 mm. A width, e.g., a diameter or cross-sectional dimension, of the container 308 range from about 40 mm to about 125 mm. For example, the container 308 may have a width ranging from about 50 mm to about 100 mm, about 50 mm to about 90 mm, about 50 mm to about 75 mm, or about 55 mm to about 65 mm. A total volume of the container 308 may range from about 10 mL to about 250 mL or from about 20 mL to about 500 mL. The kit 300 may include an empty container 308. In at least one example, the container 308 may be prefilled with a product.

Although not shown in FIG. 3, the kit 300 may further include one or more products for applying to the body, such as the skin and/or hair. The product may be a cosmetic product or a medicament. The product may be in the form of a paste, cream liquid, gel, or powder. For example, the product may have a liquid, gel, semi-liquid, semi-solid, or powder base. Exemplary products may include, but are not limited to, sunscreen, self-tanner, moisturizers, lotions, body wash, facial wash, cleansing cream, serum, exfoliator, face oil, face masks, toners, peels, body masks, body oil, body powder, hair gel, hair dye, perms, relaxers, chemical hair straighteners, rubbing alcohol, hydrogen peroxide, medical ointment, eye shadow, bronzer, highlighter, concealer, blush, lip treatment, face powder, face paint, and foundation.

FIGS. 4A-4D depict exemplary dividers that may be utilized in any of the embodiments of the present disclosure. A divider 406A having an opening 407A is shown in FIG. 4A. A divider 406B having an opening 407B is shown in FIG. 4B. A divider 406C having an opening 407C is shown in FIG. 4C. A divider 406D having an opening 407D is shown in FIG. 4D. The dividers 406A, 406B, 406C, 406D may have the same features, properties, and benefits as those described for the divider 230 of applicator system 200 and/or the divider 306 of kit 300. For example, the dividers 406A, 406B, 406C, 406D may be made from plastic, metal, rubber, sponge, or a synthetic packaging material. In some examples, the dividers 406A, 406B, 406C, 406D may be made from a flexible material. For example, the dividers 406A, 406B, 406C, 406D may be made from flexible plastic, rubber, or sponge material. Exemplary flexible materials may include neoprene, butyl rubber, nitrile rubber, silicone rubber, polyvinyl chloride (PVC), polyethylene and polypropylene. In some examples, a flexible material of the dividers 406A, 406B, 406C, 406D may provide a wiping mechanism for the brush head 216, 304a when the brush 211 is pulled out of the container 220 through the divider opening 407A, 407B, 407C, 407D.

In some embodiments, when the dividers 406A, 406B, 406C, 406D are made from a flexible material, the dividers 406A, 406B, 406C, 406D may be attached to the container 220 and the container 220 may be made from a rigid material. In other examples, when the dividers 406A, 406B, 406C, 406D are made from a flexible material, the dividers 406A, 406B, 406C, 406D may be attached to the container 220 and the container 220 may be made from a flexible material. The dividers 406A, 406B, 406C, 406D may be made from the same flexible material as the container 220 or from a different flexible material. When both the dividers 406A, 406B, 406C, 406D and the container 220 comprise a flexible material, the elasticity of the flexible material(s) may help to control the amount of product that leaves the container 220 on the brush head 216 of the brush 211. As discussed above in reference to the container 120, when the container 220 is made from a flexible material, pressure may be applied to the container 220, such as by squeezing. Squeezing the container 220 may allow inner surfaces of the container 220 to press against the brush head 216 in order to remove excess product. In some examples, pressure may be applied to both the dividers 406A, 406B, 406C, 406D and the container 220 made from a flexible material to cause deformation. For example, squeezing the dividers 406A, 406B, 406C, 406D and the container 220 may provide a wringing and wiping mechanism for the brush head 216 as the brush 211 is pulled out of the container 220. In some examples, squeezing the container 220 made from a flexible material may also apply pressure to the dividers 406A, 406B, 406C, 406D made from a flexible material.

The divider opening 407A, 407B, 407C, 407D may be large enough for the brush 211 to fit or travel through upon insertion and removal from the container 220. The divider opening 407A, 407B, 407C, 407D may be configured to receive the brush 211 without damaging the plurality of bristles of the brush head 216. In some embodiments, a diameter of the opening 407A, 407B, 407C, 407D may be larger than the width of the brush head 216. In at least one example herein, the divider 406A, 406B, 406C, 406D may be configured to conform to the shape and width of the brush head 216. For example, the divider 406A, 406B, 406C, 406D may be made from a material, such as a flexible material, which may deform at the opening 407A, 407B, 407C, 407D to receive the brush head 216. The divider 406A, 406B, 406C, 406D may fit the shape and width of the brush head 216 passing through the divider 406A, 406B, 406C, 406D. The divider opening 407A, 407B, 407C, 407D may also have a diameter that corresponds to the viscosity of the product (e.g., cosmetic product).

Divider 406A may have a portion that extends into the container 220 when attached to the container 220. For example, divider 406A may have a funnel or a conical shape. The opening 407A of the divider 406A may extend from a first end to a second end with a gradual narrowing of the diameter. For example, the first end of divider 406A may have a wider diameter or cross-sectional dimension than the second end, e.g., tapered end, of the divider 406A. The second end of the divider 406A, which may extend into the container 220, may provide a surface for removing excess product from the brush head 216 being pulled through the divider 406A, prior to reaching the first end of the divider 406A and the outside of the container 220.

In other embodiments, divider 406A may include a lid for covering the opening 407A. As shown in FIG. 4B, the divider 406B, which may correspond to the divider 406A, may include a lid 408B. The lid 408B may cover the opening 407B. As discussed above with respect to the divider 406A, the divider 406B may have a portion that extends into the container 220 when attached to the container 220. For example, divider 406B may have a funnel or a conical shape. The opening 407B of the divider 406B may extend from a first end to a second end with a gradual narrowing of the diameter. For example, the first end of divider 406B may have a wider diameter or cross-sectional dimension than the second end, e.g., tapered end, of the divider 406B. The second end of the divider 406B, which may extend into the container 220, may provide a surface for removing excess product from the brush head 216 being pulled through the divider 406B, prior to reaching the first end of the divider 406B and the outside of the container 220.

Divider 406C may also have a portion thereof that extends into the container 220 when attached to the container 220 of applicator system 200. For example, the divider 406C may fully extend beyond the surface level of the container 220 into the container 220. The divider 406C may include arms 409 that may attach to the rim of the container 220. The arms 409 may also be attached to a lower portion of the divider 406C. The lower portion of the divider 406C may provide an edge for removing excess product from the brush head 216 being pulled through the opening 407C of the divider 406C. The location of the lower portion of divider 406C within the container 220 may provide for the removal of excess product from the brush head 216 of the brush 211 prior to reaching the surface level of the container 220.

Divider 406D may be in the form of a lid or a top that covers the opening of the container 220. The opening 407D of the divider 406D may be configured as a slot or a slit in the center of the divider 406D for receiving the brush 211. The slot provided by the opening 407D of the divider 406D may include an edge for removing excess product from the brush head 216 being pulled through the divider 406D. In some examples, the opening 407D of the divider 406D may have a diameter or cross-sectional dimension that is extremely narrow to provide for edges of the opening 407D that may contact the brush head 216 being pulled through the divider 406D when the brush head 216 is flat.

The divider opening 407A, 407B, 407C, 407D may remove any excess product from the brush head 216 of the brush 211. In some embodiments, the divider 406A, 406B, 406C, 406D may have an edge or surface(s) along or near the opening 407A, 407B, 407C, 407D, which may aid in wiping, scraping, or dusting excess product from the brush 211. Further, although not depicted in FIGS. 4A-4D, the divider 406A, 406B, 406C, 406D may include additional features for removing excess product from the brush 211. The additional features may include prongs and flaps. In some examples, the additional features may be made from a flexible material. Prongs may be positioned around the circumference of the opening 407A, 407B, 407C, 407D and may extend radially into the center of the divider opening 407A, 407B, 407C, 407D through which the brush 211 travels. The prongs may be configured to contact the brush head 216 and to bend inward as the brush 211 is pulled through the divider 406A, 406B, 406C, 406D to the outside of the container 220. The inward bending of the prongs may pull any excess product contained on the plurality of bristles away from the bristles as the brush 211 is pulled in the opposite direction of the prongs. The excess product may be retained by the prongs. In at least one example, the prongs may provide a straining or wringing mechanism for the brush head 216, where the prongs push the excess product toward the inside of the container 220 as the brush 211 is pulled upward to the outside of the container 220.

Flaps may also be included as an additional feature of dividers disclosed herein and may function similar to the prongs discussed above. For instance, flaps that extend into the divider opening 407A, 407B, 407C, 407D may pull any excess product contained on the plurality of bristles away from the bristles as the brush 211 is pulled in the opposite direction of the flaps. The additional features, such as the prongs and the flaps, may narrow the opening 407A, 407B, 407C, 407D of the divider 406A, 406B, 406C, 406D.

The divider 406A, 406B, 406C, 406D may be configured to be removed or detached from the container 220 and replaced with another divider. In some examples, the divider 406A, 406B, 406C, 406D may be configured to snap onto the container 220. Dividers 406A, 406B, 406C may be configured to be popped into the container 220. To remove the divider 406A, 406B, 406C, the divider 406A, 406B, 406C may be popped out of the container 220. The divider 406A, 406B, 406C, 406D may also have a screw top configuration. In some examples, the divider 406A, 406B, 406C, 406D may be disposable. For example, the divider 406A, 406B, 406C, 406D may be disposed after using the applicator system 200. In at least one example, the divider 406A, 406B, 406C, 406D may be reusable. The divider 406A, 406B, 406C, 406D may be replaced for a different divider 406A, 406B, 406C, 406D to accommodate the brush 211 being used. For example, the divider 406A, 406B, 406C, 406D may be removed and replaced with the divider 406A, 406B, 406C, 406D that has a larger or smaller diameter for the opening 407A, 407B, 407C, 407D. The divider 406A, 406B, 406C, 406D may also be substituted with the divider 406A, 406B, 406C, 406D that is made from a different material. The divider 406A, 406B, 406C, 406D may also be replaced with a different divider 406A, 406B, 406C, 406D that is more suitable for use with the intended product. In other embodiments, the divider 406A, 406B, 406C, 406D may be permanently affixed to the container 220 and remain stationary.

FIGS. 4E-4G depict exemplary dividers that have an adjustable opening. In some embodiments, a divider as disclosed herein may include a mechanism for altering the size of the opening, such that the opening may be made smaller (e.g., narrower) or larger (e.g., wider) as desired. For example, a divider may be configured to transition to any one or more of the configurations depicted in FIGS. 4E-4G, a divider 406E having an opening 407E and a plurality of aperture blades 413E shown in FIG. 4E; a divider 406F having an opening 407F and a plurality of aperture blades 413F shown in FIG. 4F; and a divider 406G having an opening 407G and a plurality of aperture blades 413G shown in FIG. 4G. The dividers shown in 406E, 406F, 406G may be configured to obtain various diameters of the opening 407E, 407F, 407G suitable for accommodating different sizes and shapes of the brush head 216 and/or the brush 211 used with the divider 406E, 406F, 406G and/or based on the type of product deposited on the brush head 216. For example, the divider 406E, 406F, 406G may be adjusted, e.g., by twisting or rotating, to change the diameter of the opening 407E, 407F, 407G as shown in FIGS. 4E-4G. In some examples herein, the opening 407E, 407F, 407G may be adjusted to have a diameter ranging from about 4 mm to about 75 mm.

The diameter of the opening 407E, 407F, 407G may be adjusted using the plurality of aperture blades 413E, 413F, 413G. The plurality of aperture blades 413E, 413F, 413G may include two or more aperture blades 413E, 413F, 413G, such as e.g., from 5 to 9 aperture blades 413E, 413F, 413G. The plurality of aperture blades 413E, 413F, 413G may have straight edges as shown in FIGS. 4E-4G. Alternatively, the plurality of aperture blades 413E, 413F, 413G may have rounded or curved edges. The plurality of aperture blades 413E, 413F, 413G may be housed within or otherwise attached to the divider 406E, 406F, 406G, e.g., along the circumference or perimeter thereof, and may be capable of extending outward from the divider 406E, 406F, 406G into the opening 407E, 407F, 407G, upon rotating or twisting the divider 406E, 406F, 406G. Rotating the divider 406E, 406F, 406G in one direction may allow for extension of the plurality of aperture blades 413E, 413F, 413G and rotating the divider 406E, 406F, 406G in the opposite direction may allow for retraction of the plurality of aperture blades 413E, 413F, 413G. For example, turning the divider 406E, 406F, 406G in a first direction (e.g., clockwise) may cause the plurality of aperture blades 413E, 413F, 413G to extend into the opening 407E, 407F, 407G and narrow the diameter. The divider 406E, 406F, 406G may be rotated in the first direction until a desired diameter of the opening 407E, 407F, 407G is reached. Turning the divider 406E, 406F, 406G in a second direction, opposite the first direction, (e.g., counterclockwise) may cause the plurality of aperture blades 413E, 413F, 413G to retract from the opening 407E, 407F, 407G and widen the diameter. Stopping rotation of the divider 406E, 406F, 406G may cause the plurality of aperture blades 413E, 413F, 413G to lock into place. In some examples, after adjustment of the divider 406E, 406F, 406G and the opening 407E, 407F, 407G, edges of the plurality of aperture blades 413E, 413F, 413G may contact the brush head 216 as the brush 211 is pulled upward and may allow excess product to be removed from the brush 211, providing a similar effect as the prong embodiment discussed above.

The dividers 406E, 406F, 406G may have the same features, properties, and benefits as those described for the divider 230 of applicator system 200 and/or the divider 306 of kit 300 and/or the dividers 406A, 406B, 406C, 406D. For example, the dividers 406E, 406F, 406G may be made from plastic, metal, rubber, sponge, or a synthetic packaging material. In some examples, the plurality of aperture blades 413E, 413F, 413G may be made from the same material as the divider 406E, 406F, 406G. In other examples, the plurality of aperture blades 413E, 413F, 413G may be made from a different material than the divider 406E, 406F, 406G. The adjustment mechanism of the dividers 406E, 406F, 406G and the plurality of aperture blades 413E, 413F, 413G may be applicable to any divider disclosed herein. For example, any of the dividers 406A, 406B, 406C, 406D and/or divider 230 may include a plurality of aperture blades like the dividers 406E, 406F, 406G and may be adjusted to have an opening with a diameter or cross-sectional dimension similar to the opening 407E, the opening 407F, or the opening 407G shown in FIGS. 4E-4G.

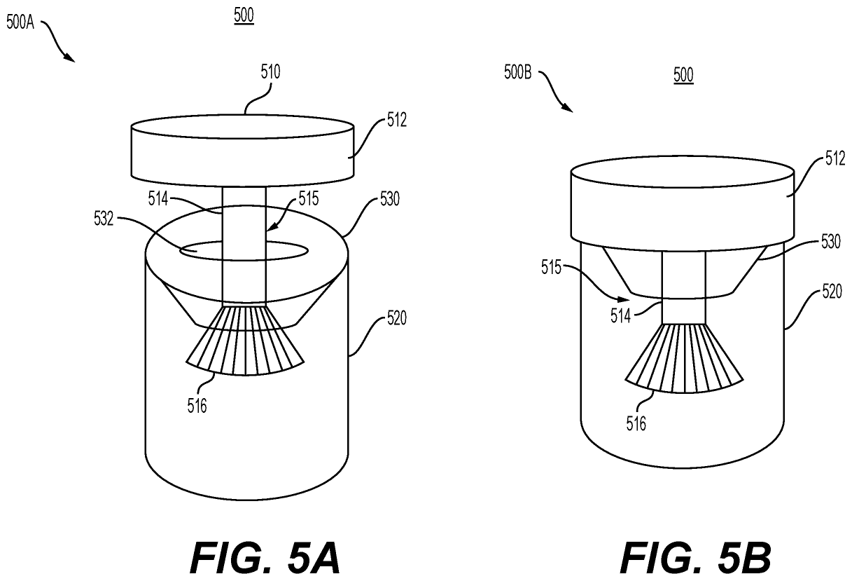

FIGS. 5A-5B depict an exemplary applicator system 500 in a first state 500A and a second state 500B, respectively. An exemplary applicator system 500 includes a brush cap 510, a container 520, and a divider 530. The brush cap 510 may include a brush 515 and a cap 512. The brush 515 may include a handle 514 and a brush head 516. The brush head 516 may comprise a plurality of bristles. The container 520 may be a refillable container. The container 520 may also include a product for applying to the body (not shown). The divider 530 may include an opening 532 and is attached to container 520. The opening 532 provides a passageway into the container 520 and out of the container 520. The opening 532 may receive and/or dispense the brush 515 of the brush cap 510. The divider 530 may be similar to the divider 406A. An upper portion of the divider 530 may be attached to the rim of the container 520 and a lower portion of the divider 530 may extend into the container 520. Further, the divider 530 may be located between the cap 512 of the brush cap 510 and the container 520.

In a first state 500A, the brush head 516 may contact the divider 530. The divider 530 may include surfaces and edges that the brush head 516 may contact in a first state. In some examples, a first state may include removal of the brush 515 of the brush cap 510 from the container 520. As the handle 514 and the brush head 516 travel through the divider 530 via the opening 532 in the first state, the divider 530 may remove any excess product contained on the plurality of bristles of the brush head 516. For example, excess product previously contained on the brush head 516 may be retained on an inner surface of the divider 530 when the brush is pulled through the opening 532 of the divider 530.

In a second state 500B, the cap 512 may be attached to container the 520 and the entire brush 515 may be housed in the container. The handle 514 may extend through the divider 530. The cap 512 may rest on a portion (e.g., upper portion) of the divider 530, which is attached to the container 520. The cap 512 may provide an airtight seal to the container 520.

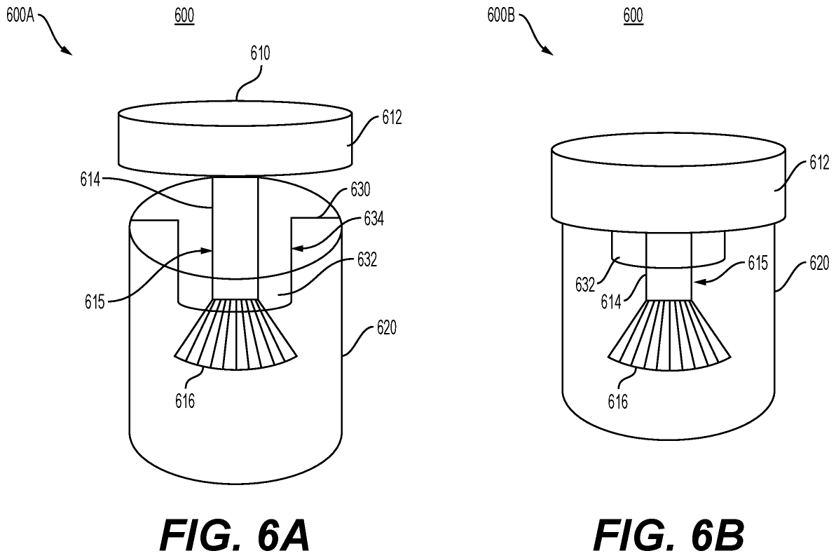

FIGS. 6A-6B depict an exemplary applicator system 600 in a first state 600A and a second state 600B, respectively. An exemplary applicator system 600 includes a brush cap 610, a container 620, and a divider 630. The brush cap 610 may include a brush 615 and a cap 612. The brush 615 may include a handle 614 and a brush head 616. The brush head 616 may comprise a plurality of bristles. The container 620 may be a refillable container. The container 620 may also include a product for that may be applied to the body (not shown). The divider 630 may include an opening 632 and may be attached to the container 620. The opening 632 may provide a passageway into the container 620 and out of the container 620. The opening 632 may receive and/or dispense the brush 615 of the brush cap 610. The divider 630 may be similar to divider 406C. Two diametrically opposed arms 634 of the divider 630 may be attached to the rim of the container 620 and a lower portion of the divider 630 may extend from the arms 634 into the container 620.

In a first state 600A, the brush head 616 may contact the lower portion of the divider 630. The divider 630 may include edges that the brush head 616 may contact in a first state. In some examples, a first state may include removal of the brush 615 of the brush cap 610 from the container 620. As the handle 614 and brush head 616 travel through the divider 630 via the opening 632 in the first state, the divider 630 may remove any excess product contained on the plurality of bristles of the brush head 616.

In a second state 600B, the cap 612 may be attached to the container 620 and the entire brush 615 may be housed in the container. The brush handle 614 may extend through the divider 630. The cap 612 may cover the arms 634 of the divider 630, which is attached to the container 620. The cap 612 may provide an airtight seal to the container 620.

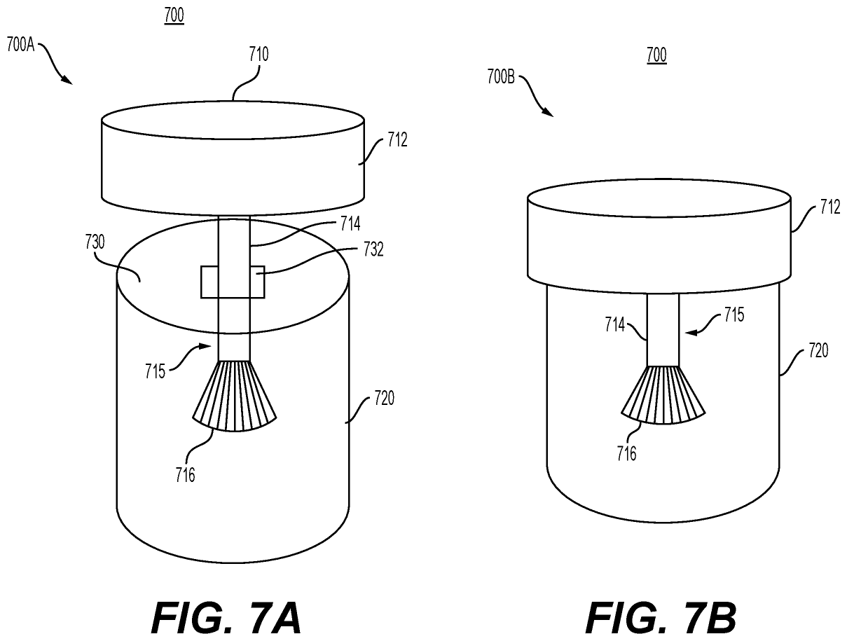

FIGS. 7A-7B depict an exemplary applicator system 700 in a first state 700A and a second state 700B, respectively. An exemplary applicator system 700 includes a brush cap 710, a container 720, and a divider 730. The brush cap 710 may include a brush 715 and a cap 712. The brush 715 may include a handle 714 and a brush head 716. The brush head 716 may comprise a plurality of bristles. The container 720 may be a refillable container. The container 720 may also include a product for applying to the body (not shown). The divider 730 may include an opening 732 and may be attached to the container 720. The opening 732 may provide a passageway into the container 720 and out of the container 720. The opening 732 may receive and/or dispense the brush 715 of the brush cap 710. The divider 730 may be similar to divider 406D. The divider 730 may be a lid that covers the opening of the container 720. The opening 732 in the center of the divider 730 may be in the form of a slot.

In a first state 700A, the brush head 716 may contact the opening 732 of divider 730. The opening 732 of the divider 730 may include edges that the brush head 716 may contact in a first state. In some examples, a first state may include removal of the brush 715 of the brush cap 710 from the container 720. As the handle 714 and the brush head 716 travel through the divider 730 via the opening 732 in the first state, the divider 730 may remove any excess product contained on the plurality of bristles of brush head 716.

In a second state 700B, the cap 712 may be attached to the container 720 and the entire brush 715 may be housed in the container 720. The handle 714 may extend through the divider 730 (not shown). The cap 712 may cover the divider 730 (not shown), which is attached to the container 720. The cap 712 may provide an airtight seal to the container 720.

FIGS. 8A-8D depict exemplary brushes 802A, 802B, 802C, and 802D, respectively. The exemplary brushes shown in FIGS. 8A-8D may be used in applicator systems and kits according to the present disclosure. Each brush 802A, 802B, 802C, and 802D may include a brush head 804A, 804B, 804C, 804D and a brush handle 806A, 806B, 806C, 806D. A ferrule 805A, 805B, 805C, 805D may connect the brush head 804A, 804B, 804C, 804D to brush handle 806A, 806B, 806C, 806D. The size of the ferrule 805A, 8056, 805C, 805D may vary based on the type of the brush 802A, 802B, 802C, 802D. In embodiments of the present disclosure, the ferrule 805A, 805B, 805C, 805D may be made from plastic, tin, aluminum, copper, brass, nickel, or chrome. The ferrule 805A, 805B, 805C, 805D may be permanently held in place to the brush 802A, 802B, 802C, 802D by crimping, swaging, or gluing to prevent damage to the brush 802A, 802B, 802C, 802D.

The brush head 804A, 804B, 804C, 804D may include a plurality of bristles. The plurality of bristles may extend through the ferrule 805A, 805B, 805C, 805D. The plurality of bristles may be made from natural or synthetic fibers to aid in the prevention of corrosion and splitting. The brush handle 806A, 806B, 806C, 806D may be made from plastic, wood, or recyclable materials.

As shown in FIGS. 8A-8D, a variety of brushes may be used in embodiments of the present disclosure. The brushes, e.g., 802A, 802B, 802C, 802D used in applicator systems herein may vary in shape and/or size. The size of the brush 802A, 802B, 802C, 802D may include the total length of the brush 802A, 802B, 802C, 802D from a first end to a second end, including the length of the brush handle 806A, 806B, 806C, 806D and the length of the brush head 804A, 804B, 804C, 804D. The size of the brush 802A, 802B, 802C, 802D may also include the width of the brush head 804A, 804B, 804C, 804D. In some aspects, the size may also include the width of the brush handle 806A, 806B, 806C, 806D. The shape of the brush 802A, 802B, 802C, 802D may refer to the shape of the brush head 804A, 804B, 804C, 804D. The brush 802A, 802B, 802C, 802D may have a total length ranging from 50 mm to 200 mm. For example, a total length of the brush 802A, 802B, 802C, 802D may range from about 50 mm to about 175 mm, from about 50 mm to about 125 mm, or from about 75 mm to about 100 mm. A width of the brush head 804A, 804B, 804C, 804D may range from about 5 mm to about 75 mm. For example, a width of the brush head 804A, 804B, 804C, 804D may range from about 5 mm to about 65 mm, from about 10 mm to about mm, from about 15 mm to about 45 mm, or from about 20 mm to about 35 mm.

The brushes of the present disclosure may also have different shapes (e.g., brush head shape). The brushes herein may have a round shape, a flat shape, a round-flat shape, a flat-top shape, a tapered (e.g., candlestick) shape, an angled shape, a fan shape, or a liner shape. The shape of the brush may be selected based on the product that is being applied.

The brush head 804A, 804C of the brushes 802A and 802C may have a tapered shape. The brush head 804B of the brush 802B may have a flat shape. The brush head 804D of the brush 802D may have an angled shape. The brush 802D may have a longer total length than the other exemplary brushes and the brush 802B may have the shortest total length of the exemplary brushes.

Brushes herein may also include other types of applicators that are capable of applying a product to the body (e.g., skin and hair), using a means other than a plurality of bristles. For example, the brush head 804C of the brush 802C may be a sponge or cotton.

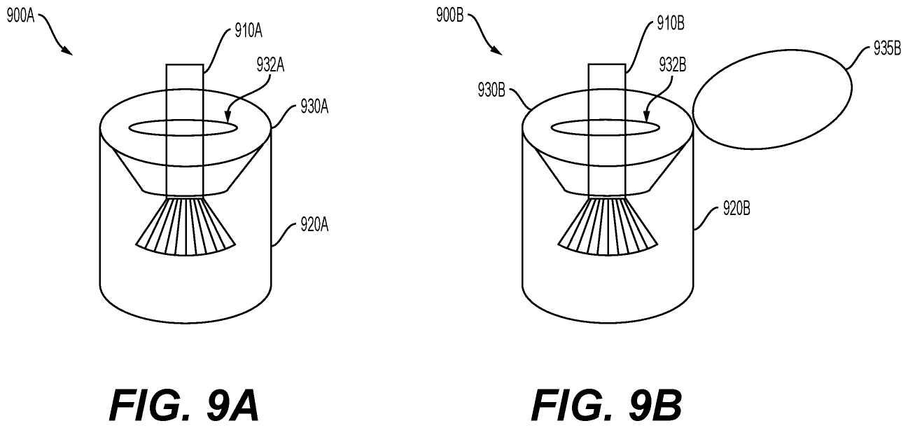

FIGS. 9A-9B depict additional exemplary applicator systems. In some applicator systems of the present disclosure, such as 900A and 900B, a brush may be free from a cap that is attached to the brush handle. Applicator system 900A may include a brush 910A, a container 920A, and a divider 930A with an opening 932A. The divider 930A may be similar to divider 406A as well as divider 530 in the applicator system 500, which is attached to the container 520. In at least one example, the applicator system 900A may be assembled from a kit that only includes the divider 930A, the container 920A, and the brush 910A.

Applicator system 900B may include a brush 910B, a container 920B, and a divider 930B. The divider 930B may be similar to divider 406B. The divider 930B may include a lid portion 935B that is attached to the divider 930B and which may cover an opening 932B of the divider 930B when in a closed position. In an open position, the lid portion 935B may expose the divider 930B and the opening 932B of the divider 930B for receiving the brush 910B. The lid portion 935B may be attached to the divider 930B via a hinge. The lid portion 935B may be used to provide an airtight seal for the container 920B. In some examples, the brush 910B may be stored in the container 920B when the lid portion 935B is closed.

In addition, any feature of an exemplary system described herein may be combined with any other features and/or described system. For example, one or more of brushes 802A, 802B, 802C, and 802D may be included and used in applicator systems 100, 200, 500, 600, 700, 900A and/or 900B.

The present disclosure also includes methods of using applicator systems. The applicator systems discloses herein may be used to apply a variety of products to the body of a user. In particular, applicator systems of the present disclosure may be used to apply a product to the skin or the hair. The method may include filling a container with a desired amount of a product for applying to the skin and/or hair. In at least one example, the method may include removing a cap connected to a brush being stored inside of the container from the top of the container in order to remove the brush from the container, prior to filling the container with the product. After the product is filled into the container, the method may include inserting a brush head of a brush connected to a cap into the container to apply the product to the brush head. Next, the method may include removing the brush from the container. In some examples, the container may include a divider attached thereto having an opening for removing excess product from the brush head. The method may further include steps of applying the product to the skin and/or hair using the brush, inserting the brush connected to the cap back into the container, and attaching the cap to the container to store the brush and seal the product within the container.

Applicator systems such as systems 100, 200, 500, 600, and 700 may be used to carry out the method steps disclosed above. The applicator systems herein may also be used in methods of removing excess product from a brush head. In some examples, the method of removing excess product from a brush head using an applicator system comprising a divider attached to a container may include steps of physically scraping, wiping, shaking, or tapping the brush head against portions of the divider to remove excess product.

Applicator systems of the present disclosure may be used in methods of applying a cosmetic product to the skin and/or hair. Various cosmetic products such as sunscreen, self-tanner, moisturizer, lotion, body wash, facial wash, cleansing cream, serum, exfoliator, face oil, face masks, toners, peels, body masks, body oil, body powder, hair gel, hair dye, perms, relaxers, chemical hair straighteners, eye shadow, bronzer, highlighter, concealer, blush, lip treatment, face powder, face paint, and foundation may be applied according to the present disclosure. In at least one example, an applicator system herein may be filled with a sunscreen and the sunscreen may be applied using the applicator system. Some methods may include a step of mixing multiple products (e.g., different cosmetics products) in a container of an applicator system prior to applying the product to the body. For example, sunscreen may be mixed with a lotion or a moisturizer.

Also disclosed herein are methods of using the applicator systems to apply a medicament to the body. Various medicaments such as rubbing alcohol, hydrogen peroxide, medical ointments (e.g., topical antibiotics and topical steroids), antiseptics, scalp treatments, pain relieving creams, chest rubs, and anesthetic creams may be applied according to the present disclosure. For example, the applicator systems and kits herein may be used to treat various ailments, injuries to the body (e.g., burns, lacerations, bruises, etc.), and skin conditions.

The applicator systems disclosed herein may provide one or more of the following effects or benefits: 1) providing a compact device for storing both a variety of products for applying to the body and a brush for applying the product; 2) removing excess product from a brush; 3) preventing certain products for application to the skin from drying out after use; 4) reducing product waste; and 5) providing a cleaner method of applying traditionally messy products to the body.

The description above and examples are illustrative, and are not intended to be restrictive. One of ordinary skill in the art may make numerous modifications and/or changes without departing from the general scope of the invention. For example, and as has been referenced, the above-described embodiments (and/or aspects thereof) may be used in combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description. The present disclosure is not limited to the exemplary shapes, sizes, and/or materials discussed herein. A person of ordinary skill in the art will recognize that additional shapes, sizes, and/or materials may be used as discussed herein to achieve the same or similar effects or benefits as discussed above. Moreover, the features of any embodiment may be used in conjunction with any of the other disclosed embodiments.

What is claimed is:

1. An applicator system comprising:
a brush including a handle and a brush head;
a cap configured to couple to a portion of the brush;
a refillable container including an opening; and
a divider, the divider including an opening for receiving the brush and removing excess product from the brush;
wherein the divider is attached to the opening of the refillable container.

2. The applicator system of claim 1, wherein in a first state, the brush is housed in the refillable container and the cap is removably attached to the refillable container.

3. The applicator system of claim 1, wherein in a second state, a brush head of the brush includes a product stored in the refillable container and is in contact with a portion of the divider.

4. The applicator system of claim 1, wherein the divider comprises a flexible material.

5. The applicator system of claim 1, wherein the refillable container has a volume of greater than 15 mL.

6. The applicator system of claim 1, wherein the brush includes a brush head and wherein the opening of the divider has a diameter that is greater than a width of the brush head.

7. The applicator system of claim 1, wherein a portion of the divider extends radially into a depth of the refillable container.

8. The applicator system of claim 1, wherein the opening of the divider has a diameter ranging from about 10 mm to about 100 mm.

9. The applicator system of claim 1, wherein the refillable container is filled with a product and the product is selected from a sunscreen, self-tanner, moisturizer, lotion, body wash, facial wash, cleansing cream, serum, exfoliator, toner, peel, mask, body oil, hair gel, hair dye, rubbing alcohol, hydrogen peroxide, medical ointment, eye shadow, bronzer, concealer, blush, lip treatment, or foundation.

10. The applicator system of claim 1, wherein the cap is a screw top, snap top, or pop-top.

11. The applicator system of claim 1, wherein the divider is fixed to the refillable container.

12. The applicator system of claim 1, wherein the divider is detachable from the refillable container.

13. The applicator system of claim 1, wherein both the refillable container and the divider comprise a flexible material.

14. The applicator system of claim 1, wherein the opening of the divider is adjustable.

15. An applicator system comprising:
a brush;
a cap configured to couple to a portion of the brush; and
a container for storing the brush and filling with a product for application to the brush;
wherein the container holds a volume of at least 20 mL of the product; and
wherein the cap attaches to an opening of the container to store the brush in the container and provide an airtight seal.

16. The applicator system of claim 15, wherein the brush is interchangeable with a different brush for storing in the container.

17. The applicator system of claim 15, wherein the brush is detachable from the cap.

18. The applicator system of claim 15, further comprising a divider attached to the opening of the container and positioned between the cap and the container, wherein the divider includes an opening sized for insertion of the brush into the container and removal of the brush from the container.

19. The applicator system of claim 18, wherein the divider is configured to remove an amount of the product from the brush when the brush is removed from the container.

20. The applicator system of claim 18, wherein both the container and the divider comprise a flexible material.

21. The applicator system of claim 18, wherein the opening of the divider is adjustable.

22. The applicator system of claim 15, wherein the container is filled with sunscreen or a medicament.

23. A kit for applying a product to hair or skin, the kit comprising:
a container for filling with a product and storing a brush for applying the product inside of the container;
the brush for applying the product; and
a divider for attaching to the container, the divider including an opening for receiving a brush and removing excess product from a brush.

24. The kit according to claim 23, wherein the kit includes a plurality of brushes and dividers.
